(19) United States
(12) Patent Application Publication
(10) Pub. No.: US 2026/0077142 A1
(43) Pub. Date: Mar. 19, 2026

(54) Exhalation-Enhanced, Nasal-Spray Apparatus for Delivering Aerosolized Liquid Substance

(71) Applicant: Aykut Unsal, JR., Blue Bell, PA (US)

(72) Inventor: Aykut Unsal, JR., Blue Bell, PA (US)

(21) Appl. No.: 18/888,126

(22) Filed: Sep. 17, 2024

(51) Int. Cl. A61M 15/08; A61M 15/00

(52) U.S. Cl. CPC A61M 15/08; A61M 15/009; A61M 15/0098; A61M 2210/0681

(57) ABSTRACT

An exhalation-enhanced nozzle apparatus constructed and arranged for use on a generic nasal spray medication container having a medication bottle, a pump assembly fixed to the bottle neck, and a removable nozzle. The apparatus has a base that envelops the pump assembly and connects in fluid communication with the pump assembly, an axially-extending nozzle, means for aerosolizing medication and expelling it through an expulsion port, and a mouthpiece into which a patient can blow to create airflow through the nozzle and out the expulsion port. The apparatus is constructed and arranged so that air blown into the mouthpiece flows through the nozzle, then mixes with and accelerates the flow rate of medication exiting the expulsion port so that the nasal medication can be delivered deeper into a patient's nasal cavity.

### FIELD OF THE INVENTION

The present invention relates to an improved apparatus for aerosolization of medication delivered throughout the nasal passages. More particularly, the invention relates to an exhalation-enhanced nozzle apparatus that can be used as a replacement for the nozzle on many generic nasal spray bottles.

### BACKGROUND OF THE INVENTION

Experiencing different sinonasal disorders is a common seasonal occurrence for many individuals and is a chronic occurrence for some. Various medications are utilized to treat sinonasal disorders including liquid medications that are injected into the patient's nasal cavity. These liquid medications are typically contained in a bottle to which some type of spray nozzle is attached, which aerosolizes and ejects the medicine from the bottle into the nasal cavity.

In their simplest form, the prior art discloses soft, plastic nasal spray bottles having an integrally-formed spray nozzle. By squeezing the bottle, positive pressure is created in the bottle, which forces the liquid medication through a tip/nozzle and ejects it in the form of a mist or spray. The dosage and expulsion rate of the medication varies greatly depending on the duration and force with which the patient squeezes the bottle. Moreover, since some prior art spray nozzles are integrally-formed with the bottle, the spray nozzle must be discarded when the medication is exhausted. Therefore, it would be desirable to provide a re-usable spray nozzle that can be used more than once with generic nasal spray bottles.

The prior art also discloses rigid plastic or glass spray bottles having a nasal spray ejector mechanism connected at its top, open end. The ejector mechanism generally includes a pump, which creates positive pressure within the bottle by depressing an actuator, and a spray tip/nozzle, which disperses the liquid as a fine mist or spray. The spray tip has a specific shape/pattern designed to deliver the spray into the nasal passages. These pumps typically deliver a predetermined dosage and expulsion rate per stroke of the actuator.

In most cases, the prior art spray nozzles create adequate aerosolization to deliver medication throughout the nasal cavity; however, delivery of the medication to the nasal passages may not be adequate for some patients whose nasal passages have an unusual anatomy, or are infected or diseased, even if the patient inhales simultaneously with injection of the medication into the nasal cavity. Therefore, it would be desirable to provide a nasal spray ejector mechanism that can more powerfully deliver the aerosolized medication into the patient's nasal passages.

### SUMMARY OF THE INVENTION

The present invention provides a nozzle apparatus that can be used as a replacement for the nozzle on many standard nasal spray bottles. The apparatus includes means for enhancing delivery of the nasal spray medication to the sinus cavities. The apparatus is constructed and arranged for use on a nasal spray medication container having a medication bottle, a pump assembly fixed to the bottle neck and bottle opening, a pump expulsion tip, and a removable nozzle. The apparatus is installed on the container after the original nozzle has been removed.

In one preferred embodiment, the apparatus generally comprises a base, a nozzle and a mouthpiece, each of which is connected in fluid communication with the medication bottle. The base has a generally-uniform construction about a central axis. A central socket is formed by an end wall extending transverse to the central axis and an axially-extending sidewall. The socket is constructed and arranged to envelop the pump assembly. A fluid-flow channel extends axially through the base end wall and is constructed and arranged to connect in fluid communication with the pump ejection tip. A shoulder projects radially from the sidewall.

The nozzle extends axially and is connected to the base at a proximal end, and has an expulsion port at a distal end. The nozzle has a medication flow channel connected in fluid communication with the base channel at the proximal end. Aerosolizing means are provided at the distal end of the medication flow channel for aerosolizing medication and expelling it through the expulsion port. An exhaust air channel extends through the nozzle to the expulsion port.

A mouthpiece is fixed at a proximal end to the nozzle and is connected in fluid communication with the exhaust air channel. The mouthpiece has an input port at a distal end into which a patient can blow to create airflow through the exhaust air channel and out the expulsion port. The mouthpiece generally comprises a tube having an input port at its distal end, a proximal end port connected to a blow port on the nozzle, and projects upwardly and outwardly relative to the nozzle. Air flowing through the exhaust air channel mixes with and accelerates the flow rate of medication exiting the expulsion port.

In one preferred embodiment, the aerosolizing means comprises a swirl chamber having a distal open end and a proximal end connected in fluid communication with the distal end of the medication flow channel. The swirl chamber induces turbulent, circular flow of the medication within the swirl chamber. Preferably, the inner diameter of the swirl chamber at the proximal end is greater than the diameter at the distal open end. In one embodiment, the swirl chamber includes a plurality of mixing fins fixed to the interior wall of the swirl chamber. In another preferred embodiment, the swirl chamber includes a plurality of grooves formed in the interior wall of the swirl chamber. In yet a further embodiment of the invention, the swirl chamber includes an insert that has a plurality of swirl channels extending axially therethrough.

The aerosolizing means preferably comprises a rod positioned within a pipe having an outer diameter smaller than the inner diameter of the pipe. The pipe is fixed co-axially within the air flow channel by a plurality of struts connecting the outer surface of the pipe to the inner surface of the nozzle. The rod is fixed co-axially within the medication channel by a plurality of splines extending from at least a portion of the outer surface of the rod. The diameter of the rod and the size of the splines are constructed and arranged so that the splined portion of the rod creates an interference fit within the pipe along an interference length but also allows medication to flow through the splines along the interference length. The flow area through the splines is less than the flow area through the pump expulsion tip. In one preferred embodiment, the splines are arranged diametrically opposed from one another, and the diameter of the rod from one spline to an opposed spline is equal to or slightly greater than the inner diameter of the pipe.

The nozzle generally comprises a tube having a proximal end port in fluid communication with the base channel, a cylindrical sidewall at a proximal end portion, a frustoconical sidewall at a distal end portion, and a blow port in the cylindrical sidewall. The mouthpiece generally comprises a tube having an input port at its distal end, a proximal end port connected to the blow port on the nozzle, and projects upwardly and outwardly relative to the nozzle.

In another preferred embodiment, the invention provides a kit comprising a plurality of exhalation-enhanced nozzle apparatuses having structural properties that are different from one another. In one preferred embodiment, the spray pattern emitted from the expulsion port of each apparatus is different to accommodate the preferences of different patients. In another embodiment, the shape and size of the base of each apparatus is different to accommodate the shape and size of different medication containers.

The present invention also provides a method of enhancing delivery of a nasal spray medication to the sinuses of a patient. The medication is contained in a spray medication container having a bottle, a pump assembly fixed to the bottle neck, a pump expulsion tip, and nozzle. The medication is aerosolized within a nozzle and expelled from the nozzle into the nasal passageway while simultaneously infusing air into the same nasal passageway. In a preferred embodiment, air is infused into the aerosolized medication before it exits the nozzle. Preferably, the air infusing step comprises the step of the patient exhausting its breath into the aerosolized medication.

### BRIEF DESCRIPTION OF THE DRAWINGS

FIG. 1A is a side elevation of a known, generic nasal spray medicine container;

FIG. 1B is a side elevation of the container shown in FIG. 1A with the spray nozzle removed to show the pump assembly;

FIG. 2 is a perspective of an apparatus in accordance with a preferred embodiment of the invention installed on a generic, nasal-spray container after the original nozzle has been removed;

FIG. 3 is an enlarged perspective of the apparatus of FIG. 2;

FIG. 4 is an enlarged, partial perspective of the apparatus of FIG. 3;

FIG. 5 is a perspective of the nozzle pipe of the apparatus of FIG. 2;

FIG. 6 is a perspective of the nozzle rod of the apparatus of FIG. 2;

FIG. 7 is a cross section of the apparatus of FIG. 2;

FIG. 7A is an enlarged, partial cross section taken from FIG. 7;

FIG. 7B is an enlarged, partial cross section taken from FIG. 7;

FIG. 8 is a cross section taken along line 8-8 of FIG. 7;

FIG. 9 is a cross section taken along line 9-9 of FIG. 7;

FIG. 10 is a cross section of the inner nozzle pipe and nozzle rod of the apparatus of FIG. 2;

FIG. 11 is a cross section taken along line 11-11 of FIG. 10;

FIG. 12 is a cross section taken along line 12-12 of FIG. 11;

FIG. 13 is a cross section taken along line 13-13 of FIG. 10;

FIG. 14 is a cross section taken along line 14-14 of FIG. 13;

FIG. 15 is a cross section taken along line 15-15 of FIG. 10;

FIG. 16 is a partial cross section of the upper portion of the nozzle taken along line 16-16 of FIG. 15;

FIG. 17 is a cross section of the upper portion of the nozzle in accordance with an additional embodiment of the invention;

FIG. 18 is perspective of the swirl chamber of FIG. 17;

FIG. 19 is a cross section of the upper portion of the nozzle showing in accordance with a further embodiment of the invention; and,

FIG. 20 is a cross section taken along line 20-20 of FIG. 19.

### DETAILED DESCRIPTION OF PREFERRED EMBODIMENTS

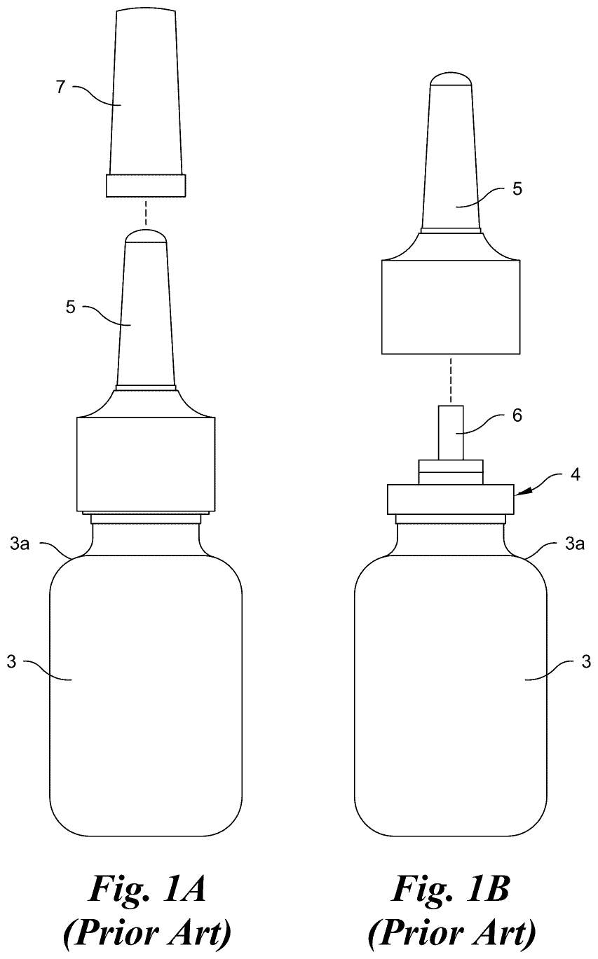

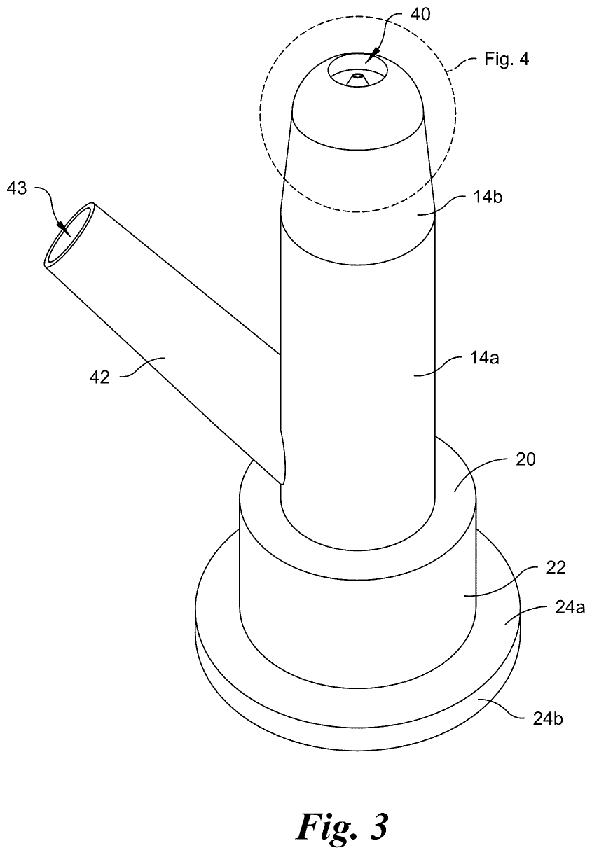

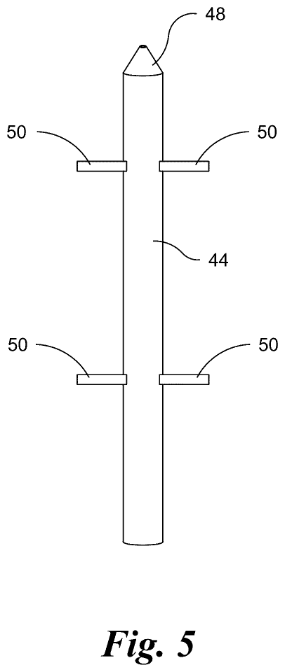

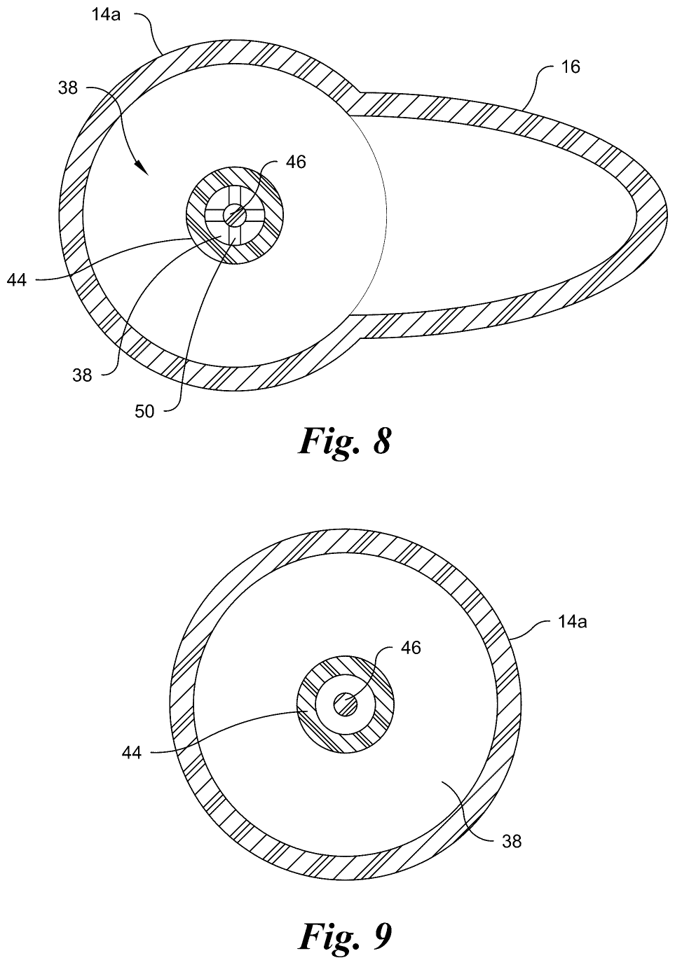

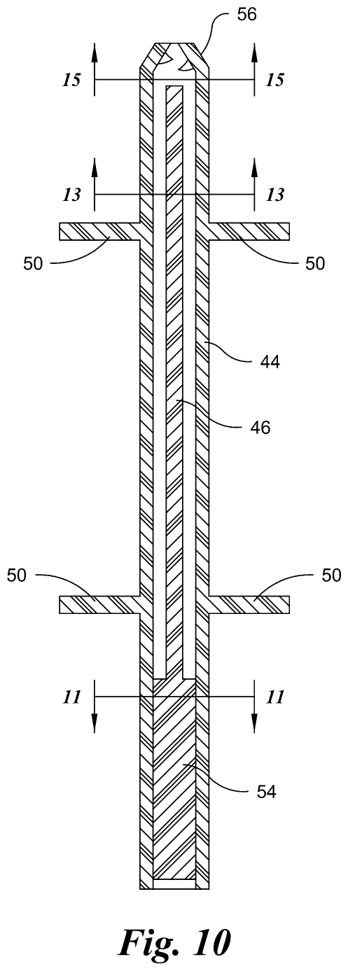

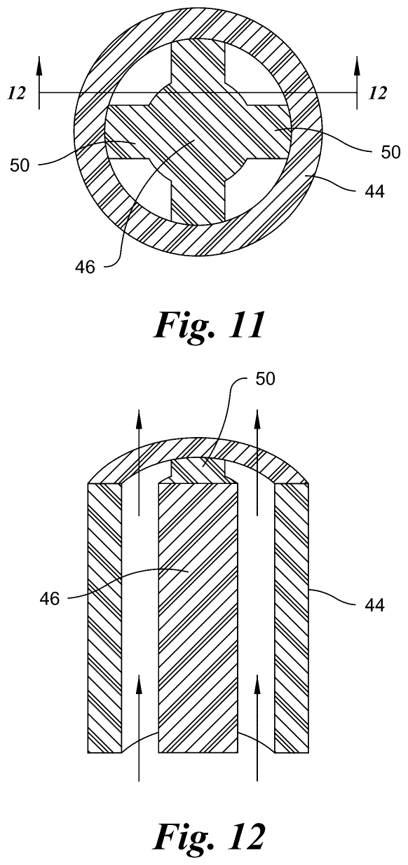

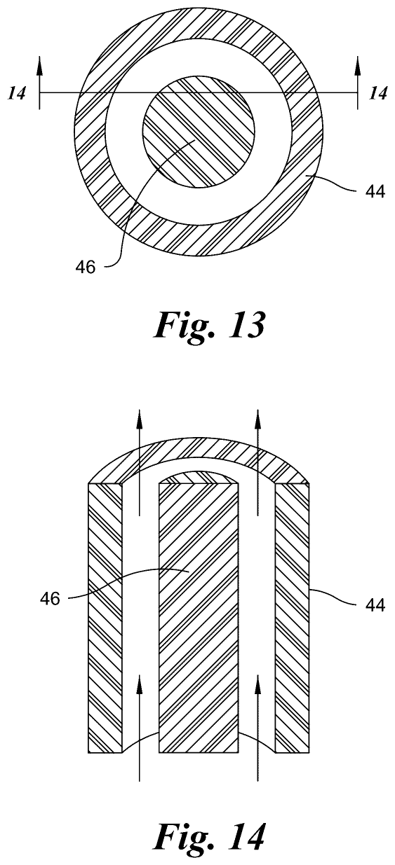

The invention is described with reference to FIGS. 1-20, and the corresponding non-limiting examples, wherein like reference numerals refer to like elements. As used herein, the terms “distal” and “proximal” are used with reference to the medication container on which the novel apparatus is installed and connected. Reference to “one embodiment” or “an embodiment” means that a particular feature, structure or characteristic described therein is included in at least one embodiment. Thus, the appearances of the phrase “in one embodiment” or “in an embodiment” appearing in various places throughout the specification are not necessarily all referring to the same embodiment.

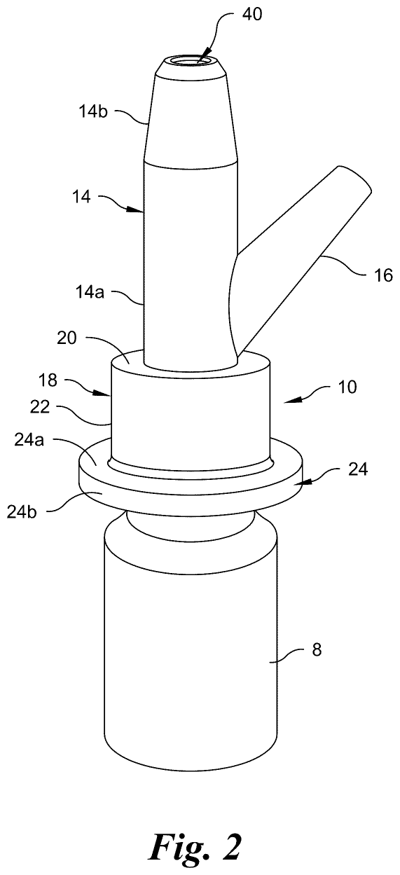

An exhalation-enhanced, nasal spray apparatus in accordance with a preferred embodiment of the invention is shown in FIGS. 2-16 and is designated generally by reference numeral 10. In preferred embodiments, the apparatus 10 is constructed and arranged to replace the nozzle 5 on a generic nasal spray medication container 2 such as shown in FIGS. 1A and 1B, which may contain, for example, a medication of fluticasone propionate nasal spray for treating rhinosinusitis. The prior art container 2 of FIGS. 1A and 1B generally comprises a glass bottle 3, a pump assembly 4 fixed to the neck and opening of the bottle 3, a nozzle 5, which engages and is connected in fluid communication with the pump assembly 4, and a cap 7 for the nozzle 5. Depression of the nozzle 5 actuates the pump assembly 4, which ejects medication from the bottle 3 through the nozzle 5.

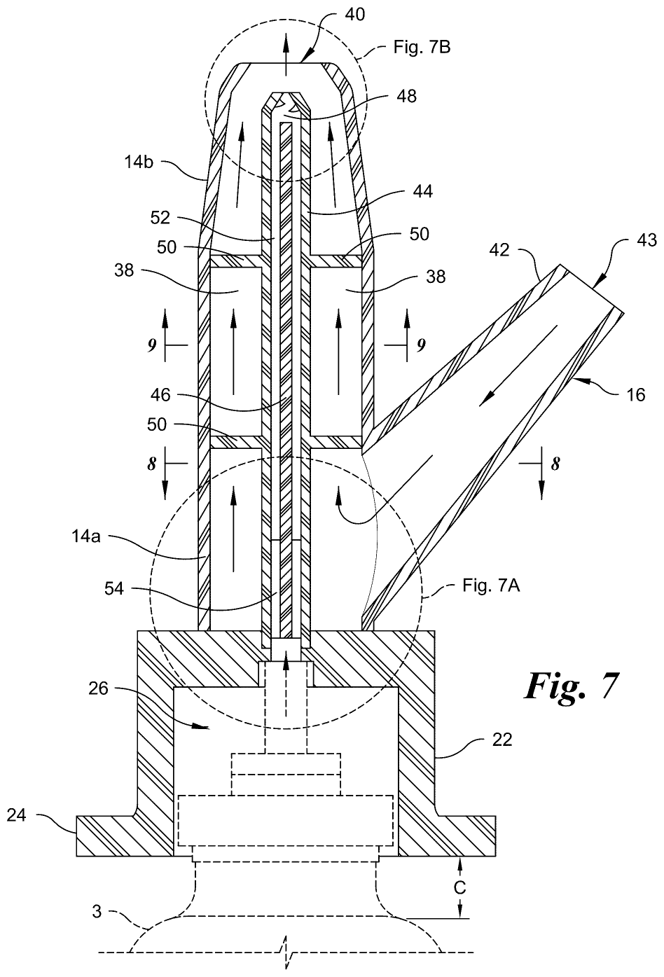

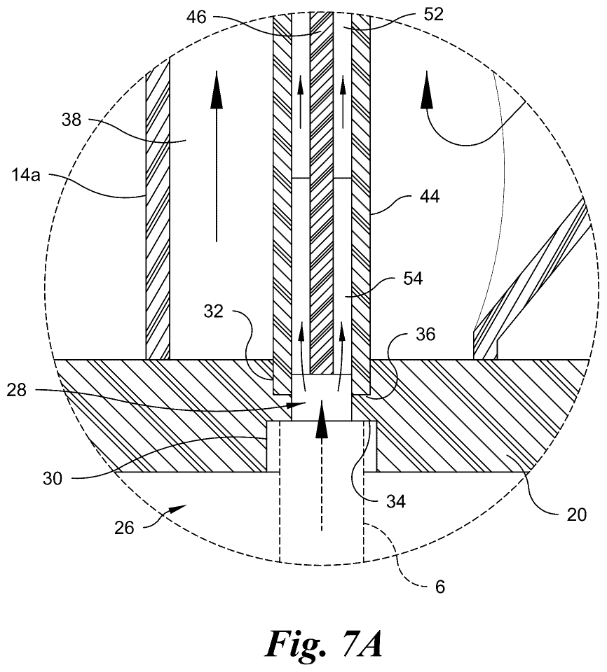

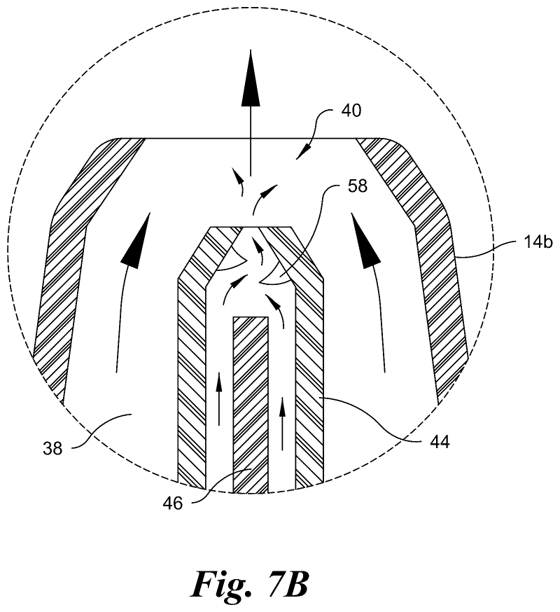

Referring to FIG. 1B, the medication container of FIG. 1A is shown with the original nozzle removed. The apparatus 10 in accordance with preferred embodiments of the invention replaces the prior art nozzle 5 by installing on and connecting in fluid communication with the pump assembly 4. As shown in FIG. 7 and described in greater detail below, the base of the apparatus 10 surrounds the pump assembly 4 with the pump ejection tube 6 connected in fluid communication with the central channel 52 of the pipe 44 within the nozzle 14.

It should be appreciated that the novel apparatus 10 can also be prepackaged with the generic medication container shown in FIGS. 1A and 1B instead of being used as a substitute for the prior art nozzle 5 shown in FIGS. 1A and 1B without departing from the scope of the invention. It should also be appreciated by those of ordinary skill in the art that the novel apparatus 10 may be used in association with medication containers having a shape, size and construction different than the container shown in FIGS. 1A and 1B without departing from the scope of the invention.

The apparatus 10 in accordance with one preferred embodiment of the invention generally comprises a base 12, a nozzle 14, and a mouthpiece 16. In FIGS. 2-15, the aforementioned components are shown as being integrally formed from a food-grade thermoplastic material such as polypropylene, polystyrene, and polyethylene terephthalate. However, it should be understood by those of ordinary skill in the art that the components could be modularly formed and assembled together.

The base 12 has a generally-cylindrical upper portion 18 formed by a distal, radially-extending end wall 20 and an axially-extending sidewall 22. A radially-extending shoulder 24 projects from the proximal end of sidewall 22. The diameter of the shoulder 24 is selected so that it provides a surface sufficiently large for a patient to hold the apparatus with opposed fingers, preferably the index and middle finger straddling the base, during use of the nozzle 14 to administer the medication. For example, in one preferred embodiment, the diameter of the shoulder 24 is about 40 mm and the portion 24a of the shoulder 24 that extends radially is about 7 mm. The thickness of the axially-extending portion 24b of the shoulder 24 need only be sufficient to create a shoulder rigid enough to grasp. In the embodiment shown in FIGS. 2-15, the thickness of the shoulder 24b is about 4 mm.

The base 12 includes a central, axially-extending, fluid-flow channel 28 through which medication from the medicine container 8 flows into the nozzle 14. Proximal 30 and distal 32 counterbores are formed in the proximal and distal sides, respectively, of the base end wall 20 co-axial with the central fluid-flow channel 28. The proximal counterbore 30 forms a shoulder 34 against which the pump ejection tube 6 impinges when the apparatus 10 is depressed on the medication container 8, thereby causing medication to flow upwardly through the central fluid-flow channel 28. In preferred embodiments, the inner diameter of the proximal counterbore 30 is slightly larger than the outer diameter of the pump ejection tube 6. The inner diameter of the central fluid-flow channel 28 in the base 12 is preferably approximately the same diameter as the inner diameter of the pump ejection tube 6 so that medication flow through the base is not restricted. The distal counterbore 32 provides a seat for the bottom of the pipe 44, described below, which sits on a distal shoulder 36 at the bottom.

The base end wall 20 and sidewall 22 form a central socket 26 into which the medication container can be inserted when the original nozzle 5 is removed. In preferred embodiments, the socket 26 is cylindrical and has a diameter “DS1” slightly larger than the diameter of the pump assembly 4 with which the apparatus 10 is intended to be used. The depth “DS2” of the socket 26 is selected so that the socket 26 envelopes the pump assembly 4 but leaves sufficient clearance “C” between the glass bottle shoulder 3a and the bottom of the base shoulder 24 for the apparatus 10 to translate upwardly and downwardly (relative to the orientation shown in FIG. 7) so that the apparatus 10 can actuate the pump assembly 4. In preferred embodiments, the clearance “C” should be at least as great as the length of the stroke of the pump assembly 4. In one preferred embodiment shown in FIGS. 2-15, the clearance “C”is about 4-6 mm.

The nozzle 14 preferably has a tubular structure with a generally cylindrical shape at its proximal end 14a, which is fixed in fluid connection to the base 12. The nozzle tapers radially inwardly at the distal end to form a frustoconical tip 14b, which inserts into the patient's nostril during use. The nozzle 14 has a central, axially-extending channel 38 connected at a proximal end in fluid communication with the central channel 28 of the base 12 and the mouthpiece 16, described below. The distal end of the channel 38 terminates at an expulsion port 40.

As best seen in FIGS. 2, 3 and 7, the mouthpiece is fixed to the outer wall of the nozzle near the proximal end 14a. The mouthpiece 16 extends distally at an angle relative to the central axis of the apparatus 10. The mouthpiece 16 comprises an elongate tube 42 having an open input port 43 at its distal end, and a proximal end connected to the outer wall of the nozzle 14 in fluid communication with the central fluid-flow channel 38 of the nozzle 14. In use, a patient exhales into the input port 43 of the mouthpiece 16, which causes air to flow down the tube 42, up the central fluid-flow channel 38, and out the expulsion port 40. In one preferred embodiment, the input port 43 of the mouthpiece 16 has an elliptical cross-section approximating the shape of a partially-opened human mouth. However, the input port 43 may have many other shapes without departing from the scope of the invention.

The length of the mouthpiece 16 should be selected so that an average-sized patient can comfortably and simultaneously insert the mouthpiece 16 into its mouth and simultaneously insert the distal end tip 14b of the nozzle 14 in its nostril. In the preferred embodiments shown herein, the mouthpiece 16 is about 3-4 cm long. As described below, exhalation of air into the input port 43 of the mouthpiece 16 enhances medication infusion deep into the patient's nasal cavity.

The nozzle 14 includes an apparatus for aerosolizing the medication just before it exits the nozzle 14. In one preferred embodiment, the aerosolizing apparatus comprises one or more of a central, axially-extending pipe 44, a coaxially-extending rod 46, and a swirl chamber 48. The pipe 44 also has a central, fluid-flow channel 52 through which the medication flows upwardly through the swirl chamber 48 and out the expulsion port 40.

The pipe 44 has a generally-cylindrical shape and is fixed at its proximal end in the distal counterbore 32 of the base 12 in fluid communication with the fluid-flow channel 28 of the base 12. The pipe 44 is positioned co-axially within the central channel 38 of the nozzle 14 by a plurality of radially-projecting struts 50 extending from the outer wall of the pipe 44 to the inner wall of the nozzle 14. As best seen in FIG. 7, in one preferred embodiment, the pipe 44 has two groups of four struts arranged 90 degrees apart from one another. The two groups of struts 50 are axially spaced along the pipe 44 at regular intervals. However, the number, spacing, angular location, and construction of the struts 50 may vary so long as the pipe is held securely within nozzle and the struts 50 do not significantly obstruct air flowing from the mouthpiece 16 through the central channel 38 in the nozzle.

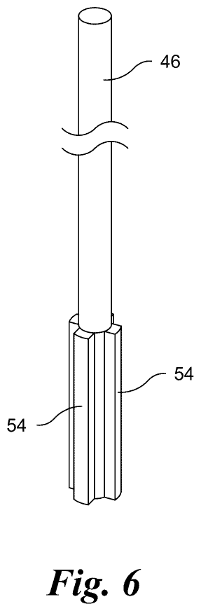

The rod 46 is solid and is positioned co-axially within the central channel 52 of the pipe 44 by a plurality of radially-projecting splines 54 extending from the outer wall of the rod 46 to the inner wall of the pipe 44. As best seen in FIG. 6, in one preferred embodiment, the rod 46 has four splines 54 arranged 90 degrees apart from one another. The splines 54 are axially spaced near the proximal end of the rod 46. The number, spacing, angular location, and construction of the splines 54 may vary so long as the rod 46 is held securely within pipe 44 and the splines 54 do not significantly obstruct the flow of medication through the central channel 52 in the pipe 44. In the preferred embodiments shown herein, the splines 54 are about 10 mm long and have a cross-sectional area of about 3.14 mm². These dimensions can be varied to create different flow velocities through this lower portion of the pipe 44.

In a preferred embodiment, the splines 54 extend from the proximal end of the rod 46 to an intermediate position approximately 20% along the length of the rod 46. In this embodiment, the proximal end of the rod 46 is positioned downstream of the proximal end of the pipe 44 so that medication flows a predefined distance within the pipe 44 before being constricted by the splines 54. In one preferred embodiment, this predefined distance may vary from 1 to 2 mm.

After the medication passes through the splines 54, it continues to flow along the rod 46 within the central fluid-flow channel 52 in the pipe 44. Because of the changing available cross-sectional fluid flow area, the medication flow velocity along the spline-less portion of the rod 46 is reduced relative to the velocity through the portion of the rod 46 with the splines 54, but is increased relative to the velocity through the central channel 28 in the base 12. In one preferred embodiment, the outer diameter of the rod 46 is about 1.3 mm and has a length of about 55 mm. The inner diameter of the pipe 44 is about 2 mm and has a length of about 58 mm. These relative diameters can be varied to create different flow velocities as the medication enters the swirl chamber 48. The length and diameter of the rod 46 are selected to create the desired exit flow properties, which are dependent on the type of medication as well as the intended patient population.

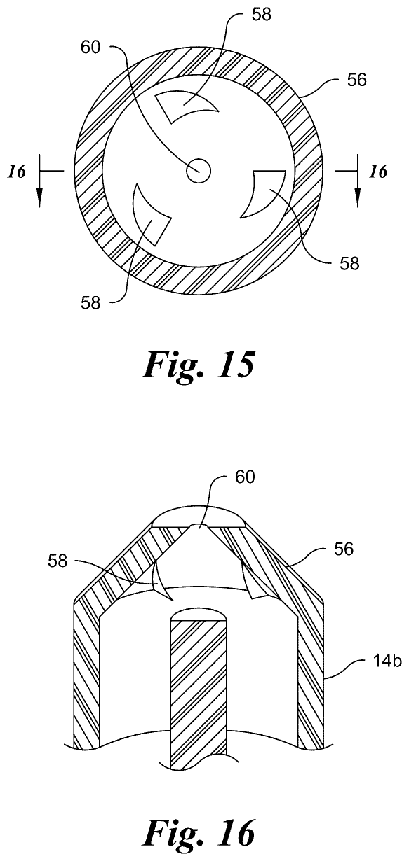

The rod 46 terminates at the entrance to the swirl chamber 48, which is constructed and arranged in fluid communication with the pipe 44 and is designed to create turbulent, non-laminar flow of the medication as part of the aerosolizing process. In one preferred embodiment, the swirl chamber 48 comprises a converging, frusto-conical chamber 56 having a plurality of radially-inwardly-projecting fins 58 as best shown in FIGS. 15-16. In one preferred embodiment, the chamber 56 is integrally formed with the walls of the pipe 44. The fins 58 are constructed and arranged to impart a swirling motion to the medication flowing therethrough. The fins 58 are spaced both axially and radially relative to one another within the swirl chamber 48 to impart the desired swirl pattern. One preferred embodiment of the fins 58 is best seen in FIGS. 7B, 15 and 16, although many different designs could be used without departing from the scope of the invention.

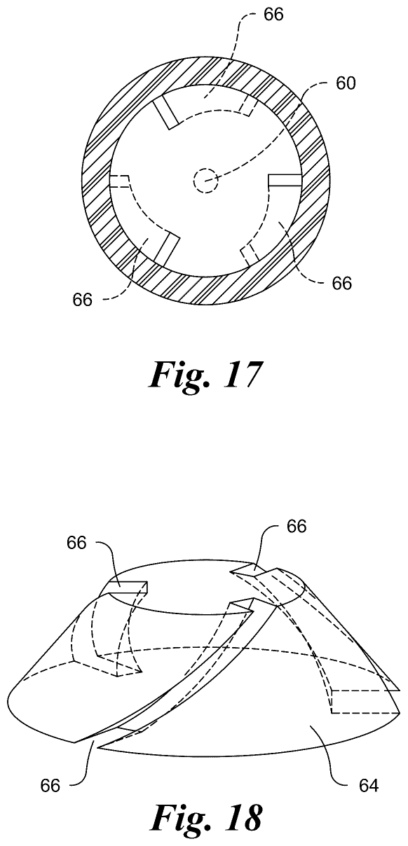

In another preferred embodiment, the swirl chamber has an insert 64 with a plurality of axially-extending fluid-flow channels 66 extending therethrough. Similar to the fins 58, the channels 66 are constructed and arranged to impart a swirling motion to the medication flowing through the swirl chamber 48. One preferred embodiment of the insert 64 is shown in FIGS. 17-18, although many different designs could be used without departing from the scope of the invention.

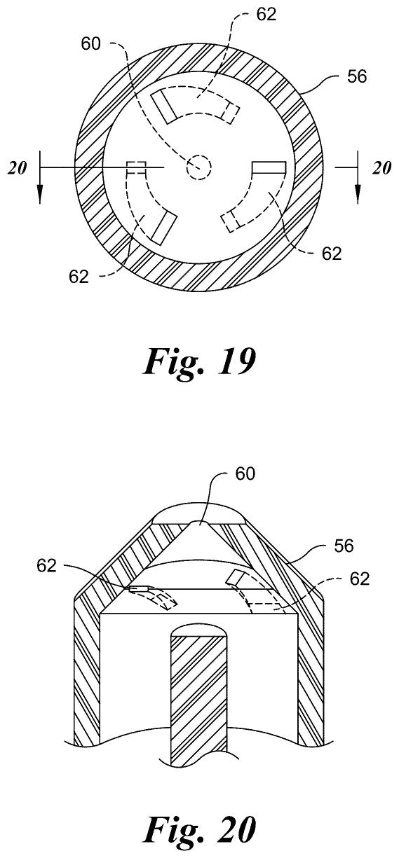

In another preferred embodiment, the swirl chamber 48 has a plurality of grooves 62 instead of fins formed on the inner surface of the swirl chamber 48. Similar to the fins 58, the grooves 62 are constructed and arranged to impart a swirling motion to the medication flowing through the swirl chamber 48. The grooves 62 are spaced both axially and radially relative to one another within the swirl chamber 48 to impart the desired swirl pattern. One preferred embodiment of the grooves 62 is shown in FIGS. 19-20, although many different designs could be used without departing from the scope of the invention.

As the medication flows through the swirl chamber 48, the flow velocity is greatly increased due to the converging shape of the swirl chamber 48. At the same time, the flow pattern is changed from linear, laminar flow through the pipe 44 to turbulent, circular flow through the swirl chamber 48. As a result, the medication is aerosolized as it exits the swirl chamber 48.

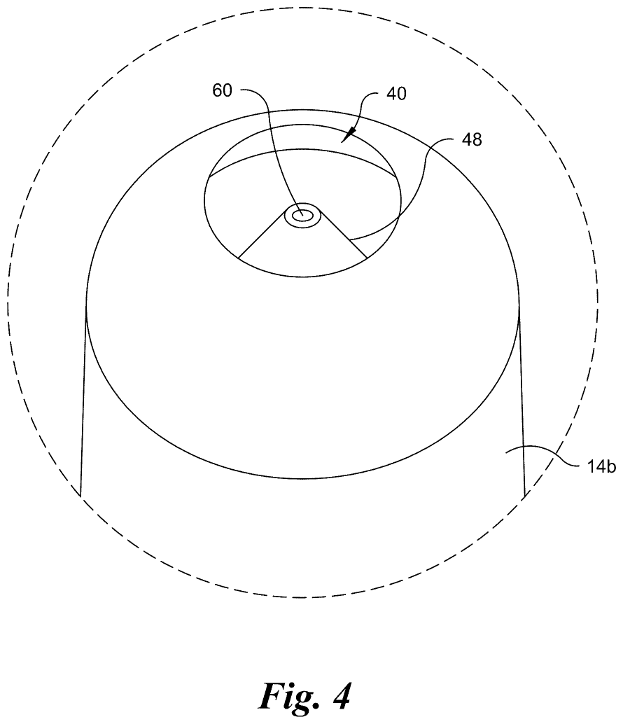

The swirl chamber 48 terminates at a distal exit port 60, which has a much smaller diameter than the diameter of the expulsion port 40 due to its function as a means for aerosolizing the medication. The exit port 60 is located within the nozzle 14 upstream of the expulsion port 40 as best seen in FIGS. 4 and 7B. In a preferred embodiment, the exit port 60 is located about 1 to 5 mm from the expulsion port 40, which allows a generous amount of exhaust air to flow around the outside of the swirl chamber 48.

As the aerosolized medication exits the swirl chamber 48, it is combined with and further accelerated by the exhaust air flowing around the swirl chamber 48 as it exits the expulsion port 40. As a result, the medication can be delivered deeper into the patient's nasal cavity than with prior art nasal spray apparatuses.

In use as a replacement for the nozzle of a generic medication container, the original nasal spray nozzle 5 is removed, which exposes the pump assembly 4 and pump ejection tube 6. The apparatus 10 is installed on the medicine container 8 with the central socket enveloping the pump assembly 4 and the pump ejection tube 6 inserted into the proximal counterbore 30 of the base 12. In preferred embodiments, the socket fits snugly enough over the pump assembly 4 to prevent the apparatus from falling off but loosely enough to allow the apparatus to translate up and down relative to the bottle. The new assembly is preferably held by straddling the base 12 with the index and middle fingers resting on the shoulder 24, while simultaneously supporting the bottom of the bottle with thumb.

To dispense the medication in the bottle in accordance with preferred embodiments of the invention, the patient inserts the nozzle 14 in one nostril and the mouthpiece 16 in its mouth. The patient then exhales into the mouthpiece 16 to initiate the flow of air down the mouthpiece 16 and up the nozzle 14. Exhaling causes the patient's throat to be closed. At the same time it is exhaling, the patient injects medication into the nozzle by depressing the apparatus 10 downwardly with its two fingers, which causes the pump injection tube 6 to translate downwardly and pump medication up the fluid-flow channel 52 in the pipe 44. As described above, the medication travels through the base 12 and the pipe 44, is aerosolized by the swirl chamber 48, combined with exhaust air near the expulsion port 40, and finally expelled at an accelerated velocity through the expulsion port 40. As the aerosolized medication enters the nasal cavity, the excess air flows around the septum and exits the opposed nostril of the patient.

The effective, repeatable delivery of a desired dose of nasal medication requires numerous factors including the viscosity and surface tension of the medication, operational characteristics of the pump assembly 4, and design of the aerosolizing means. For example, the pump assembly 4 is responsible for metered delivery of the medication dose, thereby determining the shot weight of the medication. The spray pattern, plume geometry, and droplet size distribution, which affect the bioavailability of the drug, are influenced by the combination of medication, operating parameters, and apparatus design including, but not limited to, the nozzle orifice and the swirl chamber 48. These factors can be manipulated to create a wide variety of spray patterns and spray volumes.

The nozzle 14 described above is designed to be used on a generic spray bottle, which may contain a variety of medications, and be used by a wide patient population with each patient having its own spray pattern preferences. Therefore, in a further embodiment, the invention comprises a kit having a plurality of nozzles, each of which produces a different spray pattern and/or spray volume. Each apparatus 10 includes a base 12, nozzle 14, and mouthpiece 16 to enhance expulsion from the nozzle 14 and infusion into the patient's nasal cavity. However, each nozzle 14 has different spray properties, which are generated by varying the structural properties of the aerosolization means, including the swirl chamber 48, swirl fins 58, swirl chamber exit port 60, pipe 44, rod 46, and/or splines 54, and/or other structural elements such as the mouthpiece 16 and expulsion port 40.

In another preferred embodiment, the invention comprises a plurality of apparatuses 10, each of which may be affixed to a generic medication container 2 having a removable nozzle 5. Each of the apparatuses 10 would have either different sizes and/or dimensions to affix to different sizes and/or types of generic medication containers 2. However, each apparatus 10 includes a base 12, nozzle 14, and mouthpiece 16 to enhance expulsion from the nozzle 14 and infusion into the patient's nasal cavity.

It should be emphasized that the composition of the present invention is not limited only to the specific material or specific dimensions, and has applications to other types of non-nasal medicaments, taking into consideration the desirable qualities of each material and the purposes for which the medicament is being aerosolized and injected.

Although the invention has been illustrated by reference to specific embodiments, it will be apparent that the invention is not limited thereto as various changes and modifications may be made thereto without departing from the invention scope. The various embodiments are intended to be protected broadly within the spirit and scope of the appended claims. The terms and expressions have been used as terms of description and not limitation. There is no limitation to use the terms or expressions to exclude any equivalents of features shown and described or portions thereof.

1. An exhalation-enhanced nozzle apparatus constructed and arranged for use on a nasal spray medication container having a medication bottle, a pump assembly fixed to the bottle neck and bottle opening including a pump ejection tube, and a removable nozzle, comprising:
a. a base having a generally-uniform construction about a central axis, comprising:
i. a central socket formed by an end wall extending transverse to the central axis and an axially-extending sidewall, said socket constructed and arranged to envelop the pump assembly,
ii. a base channel extending axially through said end wall constructed and arranged to connect in fluid communication with the pump ejection tube; and,
iii. a shoulder projecting radially from said sidewall;
b. an axially-extending nozzle connected to said base at a proximal end and having an expulsion port at a distal end, comprising:
i. a medication flow channel connected in fluid communication with said base channel at the proximal end;
ii. aerosolizing means at the distal end of said medication flow channel for aerosolizing medication and expelling it through said expulsion port; and,
iii. an exhaust air channel extending through said nozzle to said expulsion port;
c. a mouthpiece fixed at a proximal end to said nozzle and connected in fluid communication with said exhaust air flow channel, and having an input port at a distal end into which a patient can blow to create airflow through said exhaust air channel and out said expulsion port;
wherein said apparatus is constructed and arranged so that air flowing through said exhaust air channel mixes with and accelerates the flow rate of medication exiting said expulsion port.

2. The apparatus recited in claim 1, wherein said apparatus is constructed and arranged to replace the removable nozzle on the nasal spray medicine container.

3. The apparatus recited in claim 1, wherein said aerosolizing means comprises a swirl chamber having a distal open end and a proximal end connected in fluid communication with the distal end of said medication flow channel,
wherein said swirl chamber induces turbulent, non-laminar flow of the medication within said swirl chamber.

4. The apparatus recited in claim 3, wherein the inner diameter of said swirl chamber at the proximal end is greater than the diameter at the distal open end.

5. The apparatus recited in claim 3, wherein said medication flow channel comprises an elongate pipe, and said aerosolizing means comprises a rod positioned within said pipe having an outer diameter smaller than the inner diameter of said pipe.

6. The apparatus recited in claim 5, wherein said pipe is fixed co-axially within said air flow channel by a plurality of struts connecting the outer surface of said pipe to the inner surface of said nozzle.

7. The apparatus recited in claim 6, wherein said rod is fixed co-axially within said medication channel by a plurality of splines extending from at least a portion of the outer surface of said rod.

8. The apparatus recited in claim 7, wherein the diameter of said rod and the size of said splines are constructed and arranged so that the splined portion of said rod creates an interference fit within said pipe along an interference length but also allows medication to flow through the splines along the interference length.

9. The apparatus recited in claim 8, wherein the flow area through the splines is less than the flow area through the pump expulsion tip.

10. The apparatus recited in claim 8, wherein the splines are arranged diametrically opposed from one another, and the diameter of said rod from one spline to an opposed spline is equal to or greater than the inner diameter of said pipe.

11. The apparatus recited in claim 3, including a plurality of mixing fins fixed to the interior wall of said swirl chamber.

12. The apparatus recited in claim 3, including a plurality of grooves formed in the interior wall of said swirl chamber.

13. The apparatus recited in claim 3, including an insert in said swirl chamber that has a plurality of swirl channels extending axially therethrough.

14. The apparatus recited in claim 1, said nozzle comprising a tube having a proximal end port in fluid communication with said base channel, a cylindrical sidewall at a proximal end portion, a frustoconical sidewall at a distal end portion, and a blow port in said cylindrical side port.

15. The apparatus recited in claim 14, said mouthpiece comprising a tube having an input port at its distal end, a proximal end port connected to said blow port on said nozzle, and projecting upwardly and outwardly relative to said nozzle.

16. A kit comprising a plurality of exhalation-enhanced nozzle apparatuses constructed and arranged for use on a nasal spray medication container having a medication bottle, a pump assembly fixed to the bottle neck and bottle opening, a pump expulsion tip, and a removable nozzle, each apparatus comprising:
a. a base having a generally-uniform construction about a central axis, comprising:
i. a central socket formed by an end wall extending transverse to the central axis and an axially-extending sidewall, said socket constructed and arranged to envelop the pump assembly,
ii. a base channel extending axially through said end wall constructed and arranged to connect in fluid communication with the pump ejection tip; and,
iii. a shoulder projecting radially from said sidewall;
b. an axially-extending nozzle connected to said base at a proximal end and having an expulsion port at a distal end, comprising:
i. a medication flow channel connected in fluid communication with said base channel at the proximal end;
ii. aerosolizing means at the distal end of said medication flow channel for aerosolizing medication and expelling it through said expulsion port; and,
iii. an exhaust air channel extending through said nozzle to said expulsion port;
c. a mouthpiece fixed at a proximal end to said nozzle and connected in fluid communication with said exhaust air flow channel, and having an opening at a distal end into which a patient can blow to create airflow through said exhaust air channel and out said expulsion port;
wherein said apparatus is constructed and arranged so that air flowing through said exhaust air channel accelerates the flow rate of medication exiting said expulsion port, and
wherein the structural properties of each apparatus are different.

17. The kit recited in claim 1, wherein the spray pattern emitted from the expulsion port of each apparatus is different to accommodate the preferences of different patients.

18. The kit recited in claim 1, wherein the shape and size of the base of each apparatus is different to accommodate the shape and size of different medication containers.

19. A method of enhancing delivery of a nasal spray medication to the sinuses of a patient, comprising the steps of:
a. containing the medication in a spray medication container having a bottle, a pump assembly fixed to the bottle neck and bottle opening, a pump expulsion tip, and nozzle;
b. aerosolizing the medication within the nozzle;
c. expelling the aerosolized medication from the nozzle into the nasal passageway and simultaneously infusing air into the same nasal passageway.

20. The method recited in claim 19, wherein air is infused into the aerosolized medication before it exits the nozzle.

21. The method recited in claim 20, wherein said air infusing step comprises the step of the patient exhausting its breath into the aerosolized medication.
